(19) United States
(12) Patent Application Publication
(10) Pub. No.: US 2013/0264396 A1
(43) Pub. Date: Oct. 10, 2013

(54) MULTIDIMENSIONAL EFFECTS APPARATUS AND METHODS

(71) Applicant: Bryan Roe, La Quinta, CA (US)

(72) Inventor: Bryan Roe, La Quinta, CA (US)

(21) Appl. No.: 13/838,281

(22) Filed: Mar. 15, 2013

Related U.S. Application Data
(60) Provisional application No. 61/621,406, filed on Apr. 6, 2012.

(51) Int. Cl. B05B 12/04

(52) U.S. Cl. CPC B05B 12/04
USPC 239/11; 239/211

(57) ABSTRACT

Systems, devices, and methods for adding an entertainment dimension to a presentation are described.

### CROSS REFERENCE TO RELATED APPLICATIONS

This application claims the benefit of U.S. provisional patent application No. 61/621,406, filed Apr. 6, 2012, the entire disclosure of which is incorporated herein by reference.

### FIELD

Described herein are apparatus, systems and methods that can enhance a viewing and/or interaction experience through an additional dimension of entertainment.

### BACKGROUND

As far back as the 1860's two dimensional moving drawings were developed for entertainment. Over the subsequent century and a half, developments in cinema grew exponentially. From the introduction of sound into movies in the turn of the 20^{th} century to the 3D high definition movie experience of the early 21^{st} century, audiences are always eager for more and more entertainment.

Companies such as Disney starting in the late 20^{th} century, added various effects such as squirting water, blasting air and directing aromas to amusement park attractions and productions such as “Honey I Shrunk the Kids”, “The Indiana Jones Adventure” and “Soarin' Over California.” However, these entertainment experiences are generally effects for the masses, for example, static water effects such as sprays or mist, movable seats, and seat air “pokes.” The experiences are not individualized to sequence air and water effects to generate dynamic movement that can match an audiovisual performance.

### SUMMARY

Systems for adding an entertainment dimension to a presentation (e.g., audiovisual) are described herein. The systems can comprise at least one resource; optionally, at least one pump; at least one valve; a controller coupled to the at least one pump and the at least one valve; and at least one nozzle for expelling the resource; wherein the controller synchronizes the expulsion of the resource from the at least one nozzle with an effect. In one embodiment, the systems include at least one viewer appliance and/or viewer area such as but not limited to a chair, a stool, a couch, a bench, a walkway, a moving walkway, a viewing room, a booth, a simulation vehicle, a simulation cockpit, a simulation ship/boat bridge, or the like.

The systems include at least one resource selected from a fluid, liquid, vapor, air, compressed air, aroma, aroma oil, chilled air, heated air, chilled fluid, heated fluid, chilled liquid, heated liquid, chilled vapor, heated vapor, or a combination thereof. In some embodiments, the fluid or liquid can be water. The pump can be a fluid pump, an air pump or both. The valves can be solenoid valves or any other valves known in the art.

The controller described can be a centrally located computer, computing device and/or processor. In other embodiments, the controller can be a processor at each chair (e.g., theater seat).

Various nozzle configurations can be envisioned. In one embodiment, a plurality of nozzles is used. The plurality of nozzles can be arranged vertically, horizontally or a combination thereof. In other embodiments, a plurality of nozzles can be used in one horizontal row or vertical column. In another embodiment, a plurality of nozzles can be used in one horizontal row or vertical column and a plurality of additional nozzles in a second horizontal row adjacent to the first horizontal row. In yet another embodiment, a plurality of nozzles can be used in one horizontal row or vertical column and a plurality of additional nozzles in a second vertical column adjacent to the first vertical column. In still other embodiments, the plurality of nozzles can include two nozzles, three nozzles, four nozzles, five nozzles, six nozzles, seven nozzles, eight nozzles, nine nozzles, ten nozzles, eleven nozzles, twelve nozzles, or more.

Also described herein are systems for adding an entertainment dimension to an audiovisual presentation comprising a pressurized liquid source; a source of air; an optional pump associated with the pressurized liquid source, pressurized air source, or both; a set of valves connected to the pressurized liquid source; a set of nozzles connected to the set of valves for expelling the liquid; an air deflection device; and a controller for synchronizing with an on screen effect the expulsion of liquid from the set of nozzles and guidance of the air deflection device.

The systems include a source of liquid selected from chilled liquid, heated liquid, tap water, filtered water, filtered liquid or a combination thereof. The valves can be solenoid valves or any other valves known in the art.

The controller described can be a centrally located computer. In other embodiments, the controller can be a processor at each chair.

In some systems described herein, the air deflection device is a fan and it can pivot to deflect air in a predetermined direction.

Methods of performing an entertainment dimension during an audiovisual reproduction are also described. The methods comprise spraying liquid sequentially from two or more nozzles in a row to simulate movement across a movie screen, wherein the nozzles are controlled by a system comprising a pressurized liquid source; an optional pump associated with the liquid source; a set of valves connected to the pressurized liquid source; the set of nozzles connected to the set of valves for expelling the liquid; and a controller for synchronizing with an on screen effect the expulsion of liquid from the set of nozzles.

Also described herein are seats for encountering an audiovisual presentation comprising a first line configured to supply a pressurized liquid source; a second line configured to supply a source of air; a set of nozzles connected to the set of valves for expelling the liquid; an air deflection device; and a controller for synchronizing with an onscreen effect the expulsion of water from the set of nozzles and guidance of the air deflection device.

Also described herein are seats within a simulation automobile for experiencing an automobile simulation, such as a racing simulation or driving school experience, comprising a first line configured to supply a pressurized liquid source; a second line configured to supply a source of air; a set of nozzles connected to the set of valves for expelling the liquid; an air deflection device; and a controller for synchronizing with an effect of water expulsion from the set of nozzles and guidance of the air deflection device.

Also described herein are systems for use in conjunction with an exhibit, such as a museum exhibit. The system can comprise a first line configured to supply a pressurized liquid source; a second line configured to supply a source of air; a set of nozzles connected to the set of valves for expelling the liquid; an air deflection device; and a controller for synchronizing with an effect the expulsion of water from the set of nozzles and guidance of the air deflection device. The system can expel water, air, and or aroma that is consistent with a museum exhibit. The viewer of the exhibit can be sitting in a chair, on a bench, standing freely, standing or walking on a moving walkway while experiencing the effects added by the system.

Simulation and gaming systems are also described that include seats for encountering an audiovisual presentation. Simulations can include automobile simulations, aviation simulations, nautical simulations, and the like.

### BRIEF DESCRIPTION OF THE DRAWINGS

FIG. 1 illustrates an embodiment where a theater is fitted with a system as described herein.

FIG. 2 illustrates a liquid effect delivery system.

FIG. 3 illustrates another liquid effect delivery system.

FIG. 4 illustrates yet another liquid effect delivery system.

FIGS. 5A, 5B, 5C, and 5D illustrate different liquid nozzles and their respective effects.

FIG. 6 illustrates still another liquid effect delivery system.

FIG. 7 illustrates an air effect delivery system.

FIG. 8 illustrates a combined liquid and air effect delivery system including aroma.

FIG. 9 illustrates another combined liquid and air effect delivery system.

FIG. 10 illustrates a simulation system including an air and liquid effect delivery system.

FIG. 11 illustrates a schematic of large multi-effects system.

FIG. 12 illustrates another schematic of large multi-effects system.

### DETAILED DESCRIPTION

Described herein generally are apparatus, systems and methods for adding at least one additional entertainment dimension to a presentation. In one embodiment, the presentation can be an on screen, audiovisual production. In other embodiments, the presentation can be a still or semi-still presentation such as a museum exhibit. In other embodiments, the dimension can be added to works that may not involve a screen such as, but not limited to audio works, live musical productions, Broadway productions, dance routines, and plays. The on screen production can be a short film, a feature film, a multi-media presentation, a commercial or advertisement, a standard definition production, a high definition production, a black and white film, a color film, a two dimensional film, a three dimensional film, any other multidimensional film, a single or multiple dimension video game, a single or multiple dimension simulation, or a combination thereof.

In other embodiments, the presentation can be in the form of an automobile simulation, such as but not limited to, a racing simulation or driving school experience. Systems for use in conjunction with an exhibit, such as a museum exhibit are also described. The systems can expel water, air, and or aroma that is consistent with a museum exhibit. The viewer of the exhibit can be sitting in a chair, on a bench, standing freely, standing or walking on a moving walkway while experiencing the effects added by the system.

Generally, the systems described include at least one resource; at least one valve; a controller coupled to the at least one pump and the at least one valve; and at least one nozzle for expelling the resource; wherein the controller synchronizes the expulsion of the resource from the at least one nozzle with an on screen effect or effect of a production not involving a screen as described above.

The at least one resource selected from a fluid, liquid, vapor, air, aroma, aroma oil, chilled air, heated air, chilled fluid, heated fluid, chilled liquid, heated liquid, chilled vapor, heated vapor, or a combination thereof. In some embodiments, the fluid or liquid can be water. In some embodiments, air can be delivered from a compressed source.

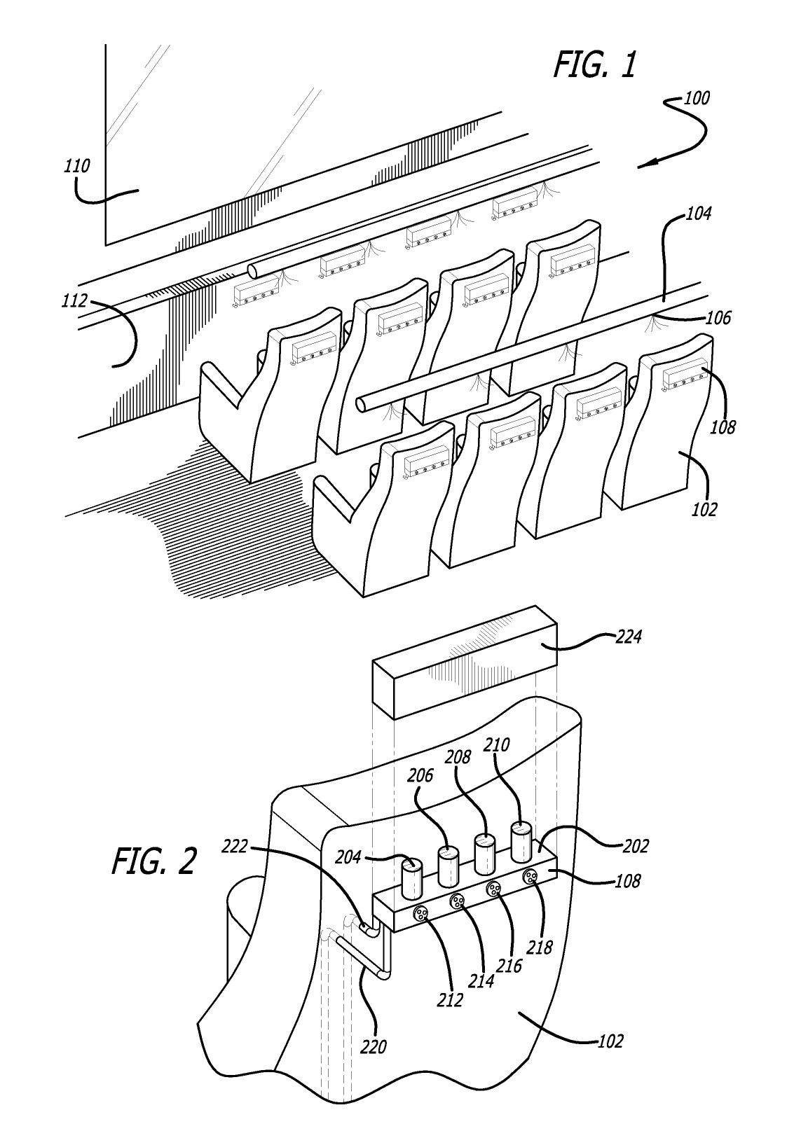

FIG. 1 illustrates aspects of the present description that can be implemented into a common movie theater. Movie theater 100 includes a plurality of seats 102, overhead resource 104 including at least one nozzle 106, and an entertainment enhancing module 108 in front of at least some of plurality of seats 102. Plurality of seats 102 can be configured as needed to view screen 110. A mounting surface 112 can be used in front of a front row of seats to provide a location to mount entertainment enhancing module 108.

As illustrated in FIG. 2, entertainment enhancing module 108 can include several components. Entertainment enhancing module 108 can include manifold 202, first valve 204, second valve 206, third valve 208 and fourth valve 210. Each valve can be coupled to a nozzle. Manifold can be formed of any suitable plastic or metal. Here, first valve 204 can be connected to first nozzle 212, second valve 206 can be connected to second nozzle 214, third valve 208 can be connected to third nozzle 216 and fourth valve 210 can be connected to forth nozzle 218. Here, a water source (not illustrated) can feed water line 220 which in turn can feed first valve 204 feeding first nozzle 212, second valve 206 feeding second nozzle 214, third valve 208 feeding third nozzle 216 and fourth valve 210 feeding forth nozzle 218. Power line 222 can provide power to first valve 204, second valve 206, third valve 208 and fourth valve 210 and a processor (not illustrated). In other embodiments, a processor is not included within entertainment enhancing module 108, but rather is located at a remote location either elsewhere at or in seat 102 or some other location in or around movie theater 100.

Entertainment enhancing module 108 can further include an optional cover 224. Cover 224 can provide protection for first valve 204, second valve 206, third valve 208 and fourth valve 210. Cover 224 can be formed of plastic or metal and can be opaque or transparent.

Although entertainment enhancing module 108 is shown with four valves and nozzles, any number of valves and/or nozzles can be used. Embodiments can include one, two, three, four, five, six, seven, eight, nine, ten or more nozzles and/or valves. In some embodiments, the more nozzles included at a given seat, the more dimension can be given to a particular audiovisual presentation. In some embodiments, valves may not be included at the module location or at each seat location. In fact, in some embodiments, a module need not be used.

On some embodiments, a grid of nozzles, supplied by appropriate resources and valves, can be used to simulate and on screen effect. For example, a grid of 3×3, 3×5, 5×3, 5×5, 6×6, 8×8, 10×10, or any combination of 100 or fewer nozzles can be used. A controller can be programmed to use various combinations and sequences of nozzles to track and/or simulate an onscreen effect. Simple linear programs can be used, but also, more complex curved, circular, curvilinear, etc. patterns can also be used.

Generally nozzles can be placed about 0.25 inches, about 0.5 inches, about 0.75 inches, about 1 inch, about 1.5 inches, about 2 inches, about 2.5 inches, about 3 inches, about 4 inches, about 5 inches or more apart horizontally or vertically.

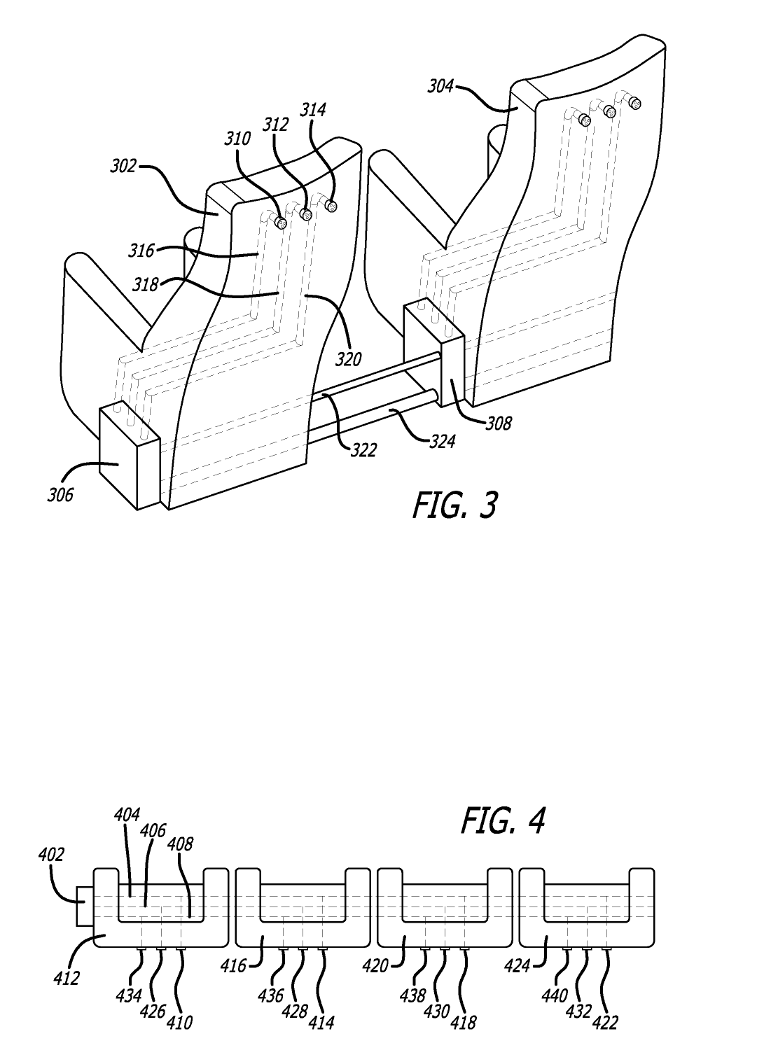

For example, as illustrated in FIG. 3 no module is included at first seat 302 and second seat 304. In some embodiments, valves are housed in first control box 306 and second control box 308, and so on. A processor(s) can also be located in first control box 306 and second control box 308. In first seat 302, first nozzle 310, second nozzle 312, and third nozzle 314 are supplied with pressurized liquid from first liquid line 316, second liquid line 318, and third liquid line 320 all from valves located within control box 306. Pressurized liquid can be supplied to control box 306 from a water source located at the end of pressurized liquid line 322.

Data and power can be fed to control box 306 through conduit 324.

In such an embodiment, any number of nozzles and valves can be used to achieve a desired result. Each control box used in a particular configuration may be identical or may be different depending on variables such as distance from nozzles to viewer, height of nozzles and the like.

A top view of an alternate configuration is illustrated in FIG. 4. Here, single control box 402 can control an entire row of seats. Control box 402 can include one or more processor and one or more valves to control liquid flows and pressures. Emanating from control box 402 can be first liquid line 404, second liquid line 406 and third liquid line 408. First liquid line 404 can feed first nozzle 410 on first seat 412, second nozzle 414 on second seat 416, third nozzle 418 on third seat 420, fourth nozzle 422 on fourth seat 424 and so on. In this way, if a valve controls the liquid pressure to first liquid line 404, similar amounts of liquid can be delivered from each of first nozzle 410, second nozzle 414, third nozzle 418, fourth nozzle 422, and so on.

Likewise, second liquid line 406 can feed fifth nozzle 426, sixth nozzle 428, seventh nozzle 430, eighth nozzle 432, and so on. Third liquid line 408 can feed ninth nozzle 434, tenth nozzle 436, eleventh nozzle 438, twelfth nozzle 440, and so on. In one embodiment, three valves in control box 402 can each independently regulate pressure/flow to first liquid line 404, second liquid line 406 and third liquid line 408.

Other rows of seats can be fed and/or controlled from an independent control box. In another embodiment, a single control box can control an entire move theater.

Liquid, in some cases in the form of water, to the various components of the presently described systems can be provided by a liquid source. Liquid in the form of water can be fed into systems described herein directly from a municipal water source of fresh water. The water can also be chilled or heated as needed to achieve a particular effect.

The liquid source can include a filter. Filtration methods can include mesh-like filters, carbon filters, UV light filtration, and the like. In some embodiments, the liquid can be filtered to remove sediment that can endanger a pumping system or clog a nozzle(s). In other embodiments, the filter can be used to clean the liquid to use around humans.

The liquid source can also include a system to introduce additives. For example, fragrances can be added to the liquid to enhance the enjoyment of the audiovisual production. For example, a fresh forest scent can be added to the liquid during a scene that is located near a forest. Further, in other embodiments, energy changing additives can be added to the liquid that can enhance water evaporation to further prevent wetting on a viewer. On the other hand, in some embodiments, an additive can be added to water that diminishes evaporation thereby allowing more wetting of a viewer. Such energy changing additives can be salts or liquids.

In some embodiments, at least one pump can be included as a component of the systems described herein. The at least one pump can reside inside a venue or in an on site maintenance room adjacent to or in the general vicinity of the venue to provide a regulated and potentially variable pressure to the system. In more elaborate systems, a central pump can be used that provides a regulated and potentially variable pressure water line to each local system. Regulated and potentially variable pressure liquid can be directed through one or more liquid lines to at least one nozzle as described above. In one embodiment, the liquid lines used are plastic and in other embodiments, the liquid lines are metal such as stainless steel or copper. In one embodiment, the liquid lines are stainless steel.

As described above, the liquid lines can include at least one nozzle. A nozzle itself can be configured as atomizing, squirting, spraying, misting, fogging, anti-wetting or a combination thereof. In any combination, a nozzle can have an appropriately sized orifice. A combination of orifice size, water pressure and/or total flow rate can allow liquid egress from nozzle(s) without wetting the viewer. Further, the nozzle can be selected such that the particle size emitted is sized appropriate for evaporation before wetting the viewer.

In some embodiments, the number of nozzles on a particular liquid line the type of nozzle used, the amount of liquid emitted from each nozzle and the like can all be dependent on the total flow rate for the liquid line. In one embodiment, the total flow rate can be about 0.05 gal/min, about 0.06 gal/min, about 0.07 gal/min, about 0.08 gal/min, about 0.09 gal/min, about 0.1 gal/min, about 0.2 gal/min, about 0.3 gal/min, about 0.4 gal/min, about 0.5 gal/min, about 0.6 gal/min, about 0.7 gal/min, about 0.8 gal/min, about 0.9 gal/min, or about 1.0 gal/min. Flow rate can also be split into a flow or flow rate per nozzle. For example, with a flow rate of 0.05 gal/min, if there were 10 nozzles on a particular line, each nozzle can provide about 0.005 gal/min of fog and/or mist.

The nozzles described herein can produce droplet sizes that may not substantially wet the intended target patron. Different droplet sizes can be achieved by varying the total flow rate, water pressure and/or the nozzle configuration. The systems described herein can create droplet sizes down to about 1 μm. In some embodiments, the systems can create droplet sizes of about 1 μm, about 2 μm, about 3 μm, about 4 μm, about 5 μm, about 6 μm, about 7 μm, about 8 μm, about 9 μm, about 10 μm, about 20 μm, about 30 μm, about 40 μm, about 50 μm, about 60 μm, about 70 μm, about 80 μm, about 90 μm, about 100 μm, about 110 μm or about 120 μm. Different ranges of the above droplet sizes can also be created. For example, a droplet size distribution of about 10 μm to about 100 μm can be achieved.

Nozzles described herein can have different geometries, orifice sizes and the like. For example, if a liquid effect of a particular shape is desired, a nozzle orifice can be cut that can allow that feature. A blade shaped orifice or specific angled orifice(s) can produce a blade of liquid effect. This blade effect can be much different than that produced by a simple round orifice.

Nozzles described herein can be mounted directly on the rear of a seat. Mounting can be flush mounted or surface mounted. Nozzles can also be mounted in a module which in turn is mounted to the rear of a seat. Nozzles can also be mounted on overhead water and/or air lies.

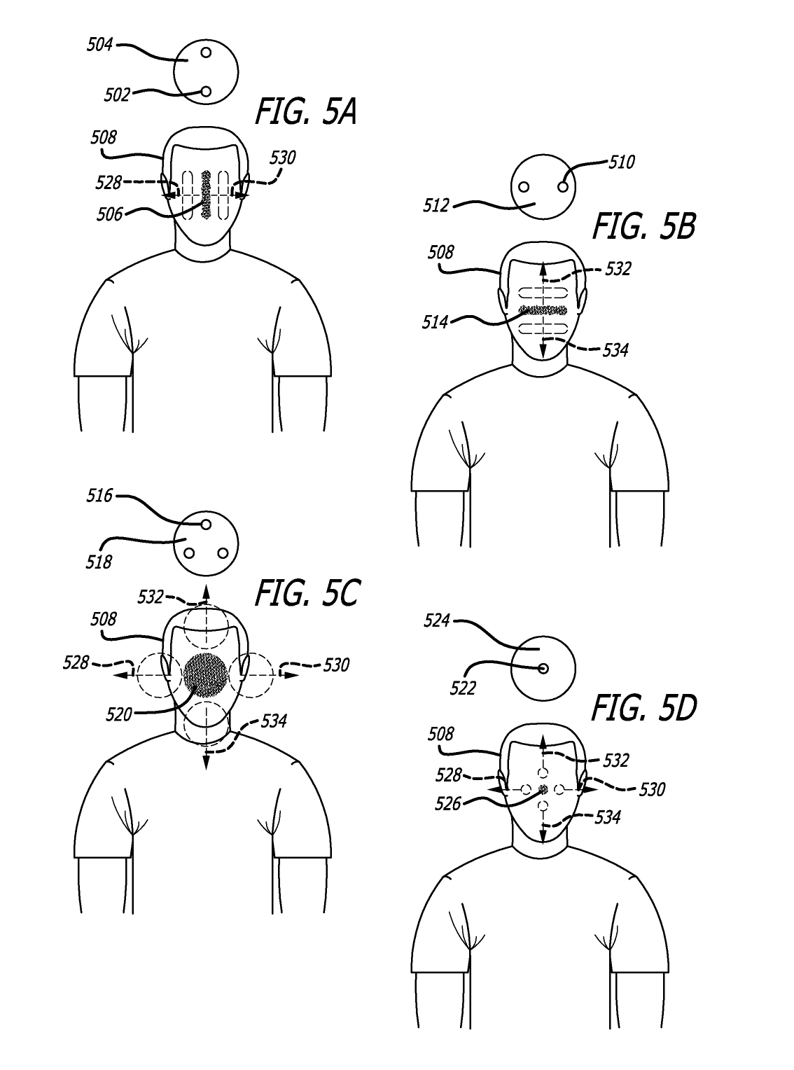

In one embodiment as illustrated in FIG. 5, various nozzle orifice or hole configurations are displayed. In FIG. 5A, two round holes 502 are vertically oriented on nozzle face 504. This configuration can produce a vertical blade 506 of water effect on viewer 508. In some embodiments, depending on water pressures and orifice size, a vertical orifice configuration can surprisingly produce a horizontal blade of water. In FIG. 5B, two round holes 510 are horizontally oriented on nozzle face 512. This configuration can produce a horizontal blade 514 of water effect on viewer 508. Again, in some embodiments, depending on water pressures and orifice size, a horizontal orifice configuration can surprisingly produce a vertical blade of water. In FIG. 5C, three round holes 516 are configured on the corners of a triangle on nozzle face 518. This configuration can surprisingly produce a large round area 520 of liquid effect on viewer 508. In FIG. 5D, one round hole 522 is placed in the center of nozzle face 524. This configuration can produce a small area 526 of liquid effect on viewer 508. Any number of nozzles of different configuration can be used with the present systems.

Also illustrated in FIG. 5A, if multiple nozzles having vertically placed orifices are oriented in a horizontal fashion and sequenced or one nozzle is movable, a vertical water effect can be moved horizontally left 528 and right 530. Likewise, but not illustrated, a vertical configuration can give a vertical effect of the same type. In FIG. 5B, if multiple nozzles having horizontal placed orifices are oriented in a vertical fashion and sequenced or one nozzle is movable, a horizontal water effect can be moved vertically up 532 and down 534. Likewise, but not illustrated, a horizontal configuration can give a horizontal effect of the same type. In FIG. 5C, if multiple nozzles having triangularly placed orifices are oriented in a horizontal fashion and sequenced or one nozzle is movable, a round water effect can be moved horizontally left 528 and right 530. Likewise, if multiple nozzles having triangularly placed orifices are oriented in a vertical fashion and sequenced or one nozzle is movable, a round water effect can be moved vertically up 532 and down 534. In FIG. 5D, if multiple nozzles having a single orifice are oriented in a horizontal fashion and sequenced or one nozzle is movable, a smaller round water effect can be moved horizontally left 528 and right 530. Likewise, if multiple nozzles having a single orifice are oriented in a vertical fashion and sequenced or one nozzle is movable, a round water effect can be moved vertically up 532 and down 534.

Any combination of horizontal or vertical movement is contemplated including combinations giving angular movements and/or shaped movements. Many nozzles can be placed on a grid to accomplish such movements. Shaped movements can include circles, ellipses, squares, triangles, rectangles, stars, curves, arches, etc.

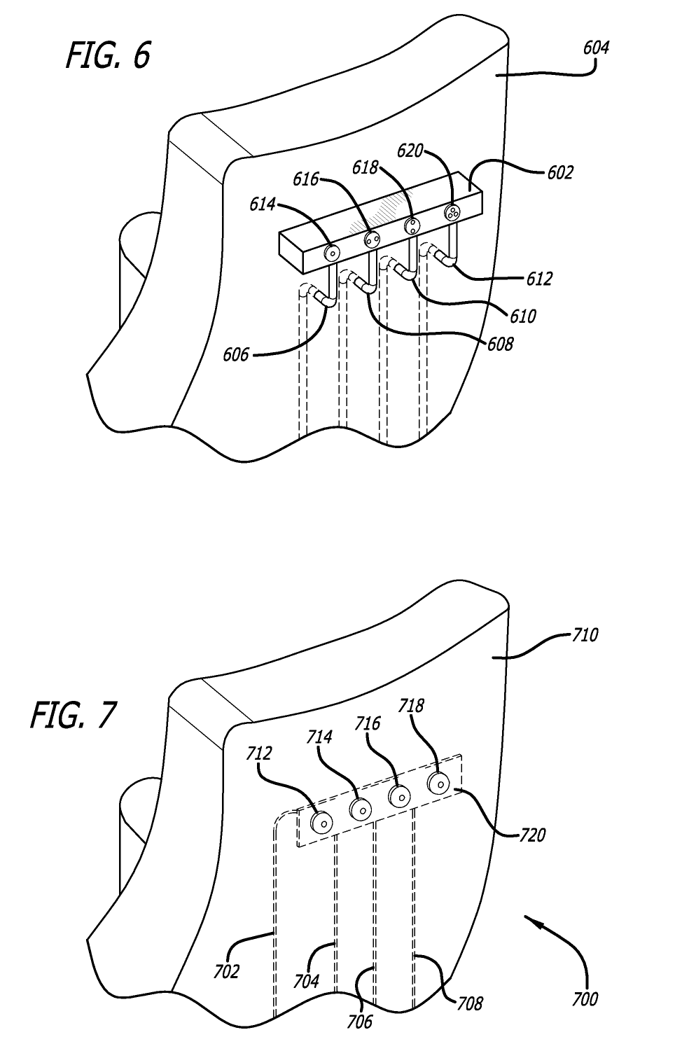

FIG. 6 illustrates four different nozzle configurations in a single housing 602. Housing 602 is mounted on the back of seat 604 and fed by first liquid line 606, second liquid line 608, third liquid line 610 and fourth liquid line 612. In this configuration, valves and processor can be located in a remote control box (not illustrated). First valve 614 has a single orifice on its face, second valve 616 has two horizontal orifices, third valve 618 has two vertical orifices, and fourth valve 620 has three corner configuration orifices. Different effects can be achieved using one or more of these nozzles at the same time or in different sequences. Nozzles can be angled to hit a viewer in a particular spot.

A housing, module, and/or set of nozzles as described herein can be mounted at an appropriate height. An appropriate height can be one that does not interfere with the viewing of the audiovisual presentation. Such a height can be about 3 feet, about 4 feet, about 5 feet, about 6 feet, about 8 feet, about 12 feet, about 16 feet, about 20 feet or more above the ground.

Nozzles can be configured in a system to emit liquid in a particular sequence. For example, if an on screen effect is to progress from left to right across the screen, if four nozzles are configured horizontally, they can be fired first nozzle, second nozzle, third nozzle, fourth nozzle. More or less nozzles can be used.

Further, each nozzle can be independently movable. In other words, each nozzle can be mounted on an actuating mount so that liquid can be directed by movement of the mount. In such a configuration, for the left to right on screen action, a single nozzle can be used to direct the liquid effect from left to right in unison with the on screen effect.

Air can also be included in the present systems to add effects. The air effects can be included in addition to the water effects, as an independent system, or used alone. In order to provide air effects, an air source can be required. An air source can include a compressed air tank supplied by an air delivery service, a rechargeable compressed air tank, or at least one fan. Compressed air tanks and rechargeable compressed air tanks (e.g. air compressor) are known in the art.

Air pressure used to add an effect can be about 1 psi, 2 about 2 psi, about 5 psi, about 7 psi, about 10 psi, about 20, psi, about 30 psi, about 40 psi, about 50 psi, about 60 psi, about 70 psi, about 80 psi, about 90 psi, about 100 psi, about 200 psi, about 300 psi, or about 400 psi.

Much like liquid effects, air effects can use air stored in one or more compressed air cylinders, delivered through air lines using valves, and ultimately emitted through one or more orifices or air nozzles. In one embodiment, the air lines used can be formed of a polymeric material such as but not limited to nylon, polyethylene, Teflon, rubber, clear plastic, colored plastic, or a combination thereof. The polymeric material can be flexible or rigid depending on the installation requirements. In other embodiments, the air lines can be metal such as stainless steel or copper.

Air nozzles, much like water nozzles described herein, can have different geometries, orifice sizes and the like. For example, if an air effect of a particular shape is desired, an air nozzle orifice can be cut that can allow that feature. A blade shaped orifice can produce a blade of air effect. This blade effect can be much different than that produced by a simple round orifice.

In one embodiment, much like liquid nozzles illustrated in FIG. 5, various air nozzle orifice configurations can be used. For example, a vertically oriented cut on an air nozzle face. This configuration can produce a vertical blade of air water effect on a viewer. Or, a horizontally oriented cut on an air nozzle face can produce a horizontal blade of air effect on a viewer. Four round holes can be configured on the corners of a square on an air nozzle face. This configuration can produce a large area of an air effect on a viewer. In a simple configuration, one round hole can be placed in the center of an air nozzle face. This configuration can produce a small or large area of air effect on a viewer depending on the orifice size. Any number of nozzles of different configurations can be used with the present air systems.

Different air effects can be achieved using one or more of these air nozzles at the same time or in different sequences. Air nozzles can be angled to hit a viewer in a particular spot. Nozzles, air and/or water, can also be changed as needed at a given location. In one embodiment, several shaped nozzles can be placed on a daisy wheel or other such device which can be controlled by a processor and changed as needed for a given effect.

Air nozzles can be configured in a system to emit air in a particular sequence. For example, if an on screen effect is to progress from left to right across the screen, if four air nozzles are configured horizontally, they can be fired first nozzle, second nozzle, third nozzle, fourth nozzle. More or less nozzles can be used.

Further, each air nozzle can be independently movable. In other words, each air nozzle can be mounted on an actuating mount so that air can be directed by movement of the mount. In such a configuration, for the left to right on screen action, a single air nozzle can be used to direct the air effect from left to right in unison with the on screen effect.

A fan can also be used to add an air effect to the on screen production. Many fan configurations are envisioned according to the present description. For example, fans can have one blade, two blades, three blades, four blades, five blades, six blades, seven blades, eight blades, nine blades, ten blades, eleven blades, twelve blades or more blades. As will be discussed in further detail, the number of blades used may depend on the particular audiovisual production and its airflow needs.

The diameter of the blades, also referred to as blade span is defined as the unit of measure from the two outermost points along the theoretical blade circumference. The diameter may vary. Some blades may have a diameter of at least 0.5 inches, 1 inch, about 2 inches, about 3 inches, or about 4 inches. The number of blades used may depend on the particular application and the amount of airflow needed.

In some embodiments, a fan may spin at a constant speed. In other embodiments, the fan can spin at variable speeds. In either case, a fan can spin at a speed of about 25 RPM, about 50 RPM, about 75 RPM, about 100 RPM, about 200 RPM, about 300 RPM, about 400 RPM, about 500 RPM, about 600 RPM, about 700 RPM, about 800 RPM, about 900 RPM, about 1,000 RPM, about 1,100 RPM, about 1,200 RPM, about 1,300 RPM, about 1,400 RPM, about 1,500 RPM, about 1,600 RPM, about 1,700 RPM, about 1,800 RPM, about 1,900 RPM, about 2,000 RPM, about 2,100 RPM, about 2,200 RPM, about 2,300 RPM, about 2,400 RPM, about 2,500 RPM, about 2,600 RPM, about 2,700 RPM, about 2,800 RPM, about 2,900 RPM, about 3,000 RPM, about 3,100 RPM, about 3,200 RPM, about 3,300 RPM, about 3,400 RPM, about 3,500 RPM, or more. The actual speed of fan rotation can depend on many factors such as, but not limited to blade span, blade pitch, blade surface area, air flow needed, noise requirements and the like. For example, larger fans with fewer blades may give off similar amounts of air as smaller fans with more blades or larger blade surfaces. Likewise, larger fans can spin at slower speeds than smaller equivalents to provide similar amounts of airflow output. One skilled in the art understands this and can choose an appropriate fan for a given output need.

Noise of a fan can be a factor in choosing a fan for a particular audio visual production. A fan as used herein can have no adverse effects on an audio visual production such as noise pollution. In some embodiments, the fans can provide ample airflow and keep noise below about 50 db, about 40 db, about 30 db, about 20 db, about 10 db, about 5 db or about 1 db.

In other embodiments, a fan can be a bladeless fan. Bladeless fans prevent buffeting which can cause choppiness in the airflow from the fan. Conversely, a bladeless fan not only supplies a constant stream of air, but because of the use of acceleration technologies, the amount of air in the resulting air stream from the fan is multiplied as compared with the air stream of an ordinary bladed fan.

A fan can include a shroud to channel its air in a particular direction or range of directions. For example, a shroud can assume a circular shape. In other embodiments, a shroud may have rectangular shapes to disperse air over a larger radial, or horizontal area than a circular shroud. In further embodiments, the shroud can shrink a fan's air channel thereby condensing and pressurizing the air before allowing the air to freely expand potentially lowering its temperature. In some embodiments, fans may not include a shroud.

A fan can freely rotate left, right, up, down and a combination thereof. For example, a fan can change direction to follow an on screen effect.

A fan can also include a safety device that can prevent tampering with or injuring oneself with the fans. In one embodiment, the safety device is a cage around the fan blades. In other embodiments, the safety device can be the blades themselves in that the blades can be made of a foam material.

FIG. 7 illustrates one embodiment of an air effects system 700. Air effects system 700 can include first air line 702, second air line 704, third air line 706 and fourth air line 708 associated with seat 710. First air line 702, second air line 704, third air line 706 and fourth air line 708 can terminate at first air nozzle 712, second air nozzle 714, third air nozzle 716 and fourth air nozzle 718 respectively all mounted on plate 720. Here, air lines are fed within seat 710, but can be located outside seat 710.

Air effects system 700 can include a control box comprising valves and a processor(s). A pressurized air line and power can be fed to the control box where air can be distributed to first air nozzle 712, second air nozzle 714, third air nozzle 716 and fourth air nozzle 718. Much like the water systems described, bursts of air can be blasted out of nozzles sequentially, variably or all at once depending on the needs of the on screen production.

In another embodiment, much like the liquid systems, plate 720 can be replaced with a housing that encases many of the components as well as the nozzles. Also, in other embodiments, the processor(s) and air source can be stored in a remote location.

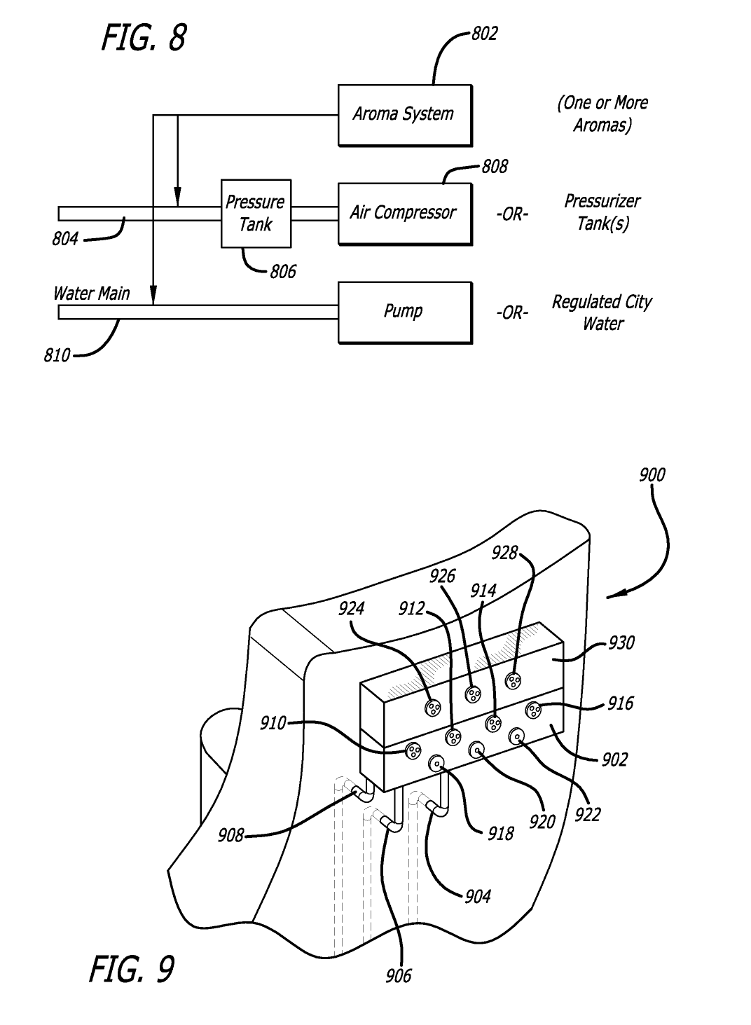

As illustrated in FIG. 8, an aroma system 802 can be used to add aroma to delivered air. Aroma can be fed into a main air line 804 or can be fed into a pressurized tank prior to delivery to the air line. If air is to be provided by air tank 806 charged by air compressor 808, when air is bled from air tank 806 into main air line 804, it can be injected with an aroma from an aroma-infused material, air passing over an aroma-infused material, an aqueous liquid, air passing over an aqueous liquid, an oil, air passing over an oil, or the like. Aroma can be added on a main air line or can be injected at a sub controller box, at a controller box located at each seat, or at a module. More than one scent may be added to an audiovisual production.

In some embodiments, aromas can be added to a liquid. Here, aroma can be fed through main water line 810 or can be injected at a sub controller box, at a controller box located at each seat, or at a module.

The systems described herein are fed power from a power source. A power source can be direct current or alternating current, and can be hardwired to the local power grid, can run off a generator, can run off a battery or batteries, can be powered by a solar panel or the like, or a combination thereof.

FIG. 9 illustrates a system 900 which includes both water effects and air effects in a single module 902. In such a system 900, a water line 904, an air line 906 and a data/power line 908 can be fed to module 902. In other embodiments, data and power can be fed in separately or even one or the other may not be needed.

Module 902 can include first water nozzle 910, second water nozzle 912, third water nozzle 914, and fourth water nozzle 916. More or fewer water nozzles can be used for a particular effect(s). Also, the water nozzles are configured horizontally, but can be configured vertically, diagonally or a combination thereof. Further, the nozzles can be different or the same. In this embodiment, they are all three triangularly configured orifices. In other embodiments, one nozzle may be a single orifice and another a blade, and the like. Each nozzle might also be independently movable.

Module 902 can also include first air nozzle 918, second air nozzle 920, and third air nozzle 922. Like the water nozzles above, more or fewer air nozzles can be used for a particular effect(s). Also, the air nozzles are configured horizontally, but can be configured vertically, diagonally or a combination thereof. Further, the air nozzles can be different or the same. In this embodiment, they are all single orifices. In other embodiments, one nozzle may be a single orifice and another a blade, and the like. Each nozzle might also be independently movable. Also, the air portion of system 900 can be replaced or augmented with one or more fans. For example, first fan 924, second fan 926 and third fan 928 can be mounted on module 902 or on cover 930. Here, they are located on cover 930. More or fewer fans can also be used and each can be independently moveable.

As in other embodiments, a control box can be located at each seat or can be more centrally located. Also, valves and/or processors can be located within or on module 902 or can be located in a control box.

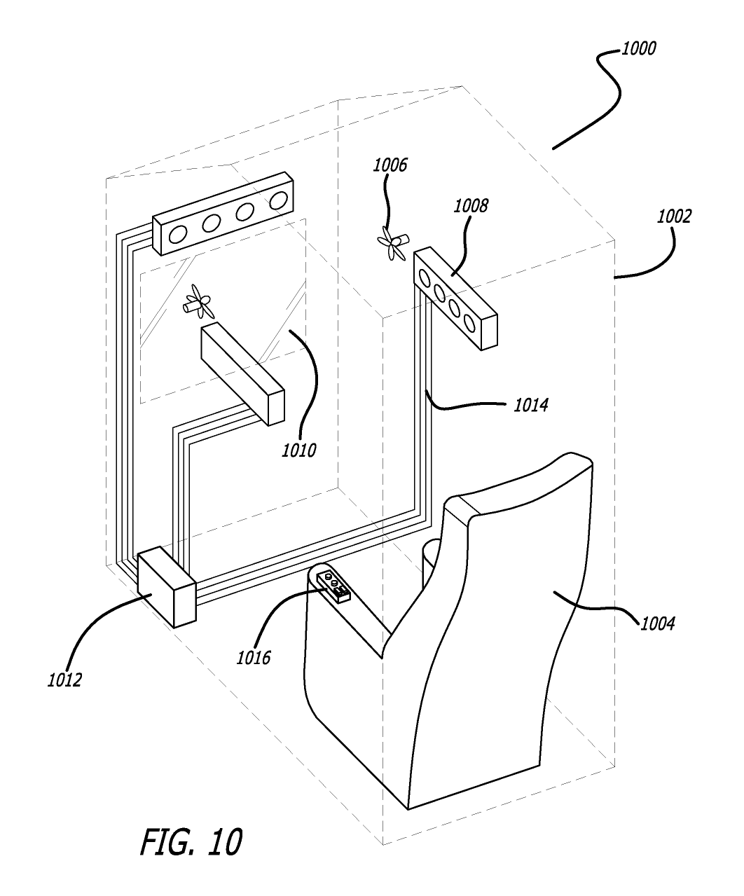

In one embodiment, the systems described can be used in a simulator and/or video game. FIG. 10 illustrates simulation system 1000. As one skilled in the art can envision, simulation systems can be modified and/or used for enjoyment and/or training.

Simulation system 1000 includes an optional enclosure 1002, at least one seat 1004, at least one air effect source 1006, at least one water effect source 1008, a screen 1010, a control box 1012, source lines 1014 and a controller 1016. A simulator system can include one seat or multiple seats depending on the game or simulation to be performed or interacted with. Here, air effect sources are fans, but can easily be replaced with compressed air and nozzle systems as described herein. Also, three water effects sources are used, one in front of the user, and one on each side. A further water effects source can also be located behind the user.

Controller 1016 can be of any kind necessary for the simulation and/or game being performed or displayed. For example, in a flying adventure, a yolk may be used, in a racing game a steering wheel and stick shift may be used, in a hunting adventure, a gun may be used, or in a video game, a game console controller may be used. If the system has no user input and/or is not interactive, no controller 1016 may be needed.

In some embodiments, seat 1004 is not needed. For example, in a combat simulator, the participant can be standing. Motion detection systems can be employed to track the users position and the effects elements can move relative to the user to add position specific effects as needed. Such detection systems include global positioning, camera detection, and the like.

Simulation systems, games, and displays for which system 1000 may be useful include, but are not limited to flight simulators, racing simulators, nautical simulators, interactive sports games, combat simulators, off road adventures, a movie, a police training tool, and the like.

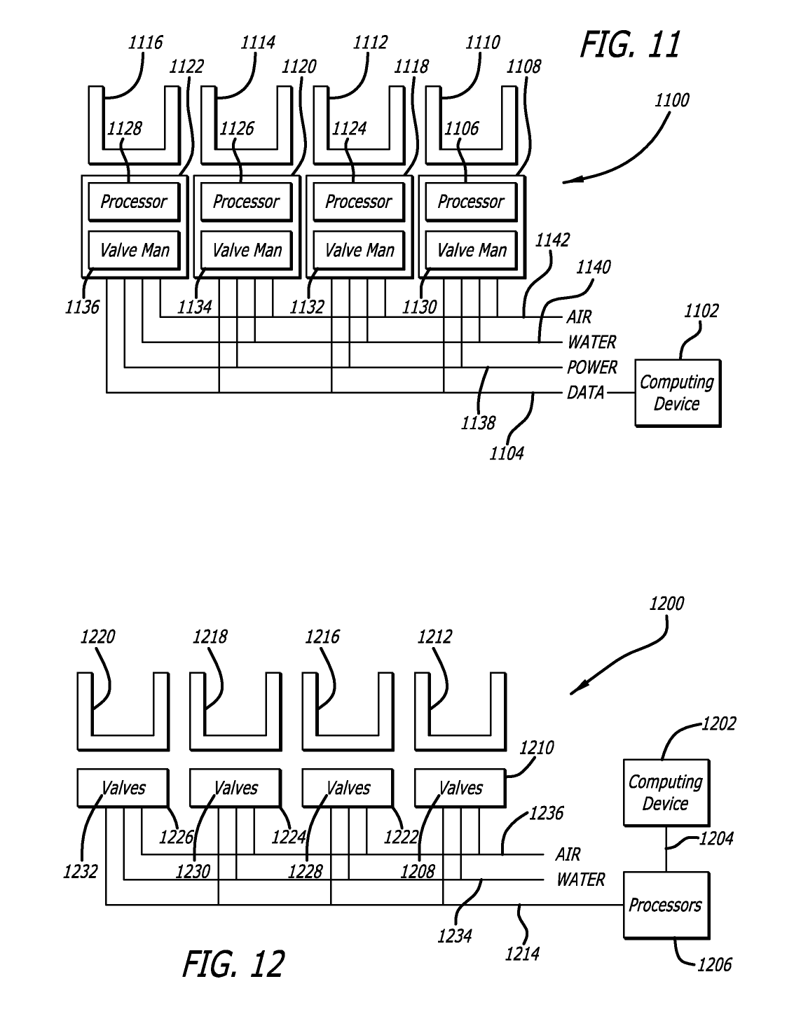

A system according to the present disclosure can be illustrated as in FIG. 11. In system 1100, a remote computing device 1102 provides data through data line 1104. Data can be provided to a processor 1106 within a control box 1108 located at each seat 1110. Second seat 1112, third seat 1114, fourth seat 1116 and so on each have a control box (e.g. second control box 1118, third control box 1120, fourth control box 1122, and so on) including a processor (second processor 1124, third processor 1126, fourth processor 1128, and so on). Each processor can receive the same data from remote computing device 1102 or can receive unique data. Each processor at each seat can control a valve manifold. For example, processor 1106 can control valve manifold 1130, second processor 1124 can control second valve manifold 1132, third processor 1126 can control third valve manifold 1134, fourth processor 1128 can control fourth valve manifold 1136, and so on.

Also, system 1100 can also include a power line 1138 that can provide power to each processor, control box and/or each valve manifold. In some embodiments, power and data can be combined into one line using such protocols as power over ethernet (PoE).

System 1100 can also include one or both of water line 1140 and air line 1142. Water line 1140 and/or air line 1142 can be connected to each valve manifold in each respective control box. Delivery duration of air and/or water can be control by each processor which in turn can receive commands from remote computing device 1102. In some embodiments, air can be provided by one or more fans at each seat location. Each set of fans can be controlled by its processor, again receiving commands from remote computing device 1102.

Another system according to the present description can be illustrated as in FIG. 12. In system 1200, a remote computing device 1202 provides data through data line 1204 to a remote processor bank 1206. Data can then be provided to a valve manifold 1208 within a control box 1210 located at seat 1212 through transfer data line 1214. Second seat 1216, third seat 1218, fourth seat 1220 and so on each have a control box (e.g. second control box 1222, third control box 1224, fourth control box 1226, and so on) including a valve manifold (second valve manifold 1228, third valve manifold 1230, fourth valve manifold 1232, and so on). Each processor within remote processor bank 1206 can receive the same data from remote computing device 1202 or can receive unique data. Each processor can then control a valve manifold. In some embodiments, remote processor bank 1206 may not be required and remote computing device 1202 can provide identical data to each valve manifold.

Also, system 1200 can also include a power line (not illustrated) that can provide power to each control box and/or each valve manifold. In some embodiments, power and data can be combined into one line using such protocols as PoE.

System 1200 can also include one or both of water line 1234 and air line 1236. Water line 1234 and/or air line 1236 can be connected to each valve manifold in each respective control box. Delivery duration of air and/or water can be controlled by each processor which in turn can receive commands from remote computing device 1202. In some embodiments, air can be provided by one or more fans at each seat location. Each set of fans can be controlled by its processor, again receiving commands from remote computing device 1202. In some embodiments, air can be provided by one or more fans at each seat location. Each set of fans can be controlled by a remote processor, again receiving commands from remote computing device 1102.

Although system 1100 and system 1200 can achieve the same results, use of either system can depend on a particular venue where the system is to be installed and implemented. For example, although system 1100 can appear to be more complex, problems can be solved by simply replacing a control box whereas in system 1200, a control box and separate remote processor may need replacing. However, system 1200 may be more cost effective to install as each seat does not need an embedded processor within the control box. A skilled artisan can weigh the options of any of the systems described herein including system 1100 and system 1200 and adapt a system for a particular venue.

Methods of performing an entertainment dimension during an audiovisual reproduction can be achieved using the systems described. The methods comprise spraying water sequentially from two or more nozzles in a row to simulate movement across a movie screen, wherein the nozzles can be controlled by a system comprising a pressurized source of water; an optional pump associated with the water source; a set of valves connected to the water source; the set of nozzles connected to the set of valves for expelling the water; and a controller for synchronizing with an on screen effect the expulsion of water from the set of nozzles. Each component of the method can be varied using the components described herein.

### Example 1

### Adding an Entertainment Dimension to a Surfing Scene

A module as described herein is placed in front of a theater seat. The module includes four nozzles placed horizontally each spaced about two inches apart. Each nozzle is fed by a pressurized water line each including an independent valve. A controller opens and closes the valves.

An action movie is displayed on screen. During a particular scene, a surfer glides on a wave across the screen from left to right. The controller is pre-programmed to expel water from the four valves in sequence from left to right as the surfer glides across the screen. As the surfer glides across the screen, the left most nozzle expels a short burst of water, followed by the second left most nozzle, followed by the third left most nozzle, and finally the right nozzle. As the surfer glides across the screen, the viewer feels as if water is hitting them as a result of the passing surfer.

### Example 2

### Adding Another Entertainment Dimension to a Firefighting Movie Scene

A module as described herein is placed in front of a theater seat. The module includes a single movable nozzle placed in the center. The nozzle is fed by a pressurized water line including a valve. A controller opens and closes the valve. The movable nozzle is on a programmable solenoid that can direct the nozzle in multiple directions.

A firefighter documentary movie is displayed on screen. During a particular scene, a firefighter putting out a house fire projects his hoses water stream across the screen from left to right. The controller is pre-programmed to expel water from the valve and move the nozzle to direct water from the right-to-left in sequence as the firefighter directs his hose water across the screen. As the firefighter expels water across the screen, the viewer fells as if water is hitting them as a result of the passing hose water.

### Example 3

### Adding Multiple Entertainment Dimensions to an Action Movie Scene

A module as described herein is placed in front of a theater seat. The module includes a single movable nozzle placed in the center. The module includes four nozzles placed horizontally each spaced about two inches apart. Each nozzle is fed by a pressurized water line each including an independent valve. A controller opens and closes the valves. The module also includes three 4 inch fans spaced about 2 inches apart in a horizontal configuration. The fans can each spin at a maximum of about 2,500 RPM.

An action movie is displayed on screen. During a particular scene, a hero is driving a jet ski and being chased by villains. Instantly, a second villain boat races across the screen from right to left in front of the hero's jet ski. As the hero is moving in the jet ski, the fans can blow at the viewer giving a sensation of moving on the jet ski. The controller is pre-programmed to expel water from the four valves in sequence from right to left as the boat races across the screen. As the boat races across the screen, the right most nozzle expels a short burst of water, followed by the second rightmost nozzle, followed by the third rightmost nozzle, and finally the left nozzle. As the boat races across the screen, the viewer feels as if water is hitting them as a result of the passing boat. Additionally, a seawater aroma can be added to the water or to the air being directed at the viewer.

### Example 4

### Adding an Entertainment Dimension to a Racing Simulation

A system as illustrated in FIG. 10 is provided. Seat 1004 from FIG. 10 is replaced by a seat within a simulated race car. An interactive racing scene is displayed on screen. When an accident occurs in front of the simulated race car, for example, a tire flashes past the car and an air effect passes across the users face. Also, the controller is pre-programmed to expel water from the valve toward the user when the race car encounters rain or fog, or crosses or passes through water on the course.

### Example 5

### Adding an Entertainment Dimension to a Static Museum Exhibit

A moving walkway is provided through a museum exhibit. In one room through which the moving walkway passes there exists an exhibit of a pre-historical display of dinosaurs. As a pterodactyl passes close by the viewers and an air effect moves past the viewers to add an additional dimension of effect. An aroma can also be added to the air feature.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

I claim:

1. A system for adding an entertainment dimension to a presentation comprising:
at least one resource;
at least one valve;
a controller coupled to the at least one valve; and
at least one nozzle for expelling the resource;
wherein the controller synchronizes the expulsion of the resource from the at least one nozzle with an effect.

2. The system according to claim 1 further comprising at least one pump.

3. The system according to claim 1 wherein the at least one resource is selected from fluid, liquid, vapor, chilled fluid, heated fluid, chilled liquid, heated liquid, chilled vapor, heated vapor or a combination thereof.

4. The system according to claim 1 wherein the at least one pump is a water pump.

5. The system according to claim 1 wherein the at least one valve is a solenoid valve.

6. The system according to claim 1 wherein the controller is a centrally located computing device, computer, processor, processing device, or a combination thereof.

7. The system according to claim 1 wherein the presentation is a movie, a film, a museum exhibit, a simulation, or an on-stage production.

8. The system according to claim 1 wherein the system comprises two or more nozzles in one horizontal row or vertical column.

9. The system according to claim 8 wherein the system comprises two or more additional nozzles in a second horizontal row adjacent to the first horizontal row.

10. The system according to claim 8 wherein the system comprises two or more additional nozzles in a second vertical column adjacent to the first vertical column.

11. A system for adding an entertainment dimension to a presentation comprising:
a pressurized liquid source;
a set of valves connected to the pressurized liquid source;
a set of nozzles connected to the set of valves for expelling a liquid from the pressurized liquid source; and
a controller for synchronizing with an effect the expulsion of the liquid from the set of nozzles.

12. The system according to claim 11 wherein the source of liquid is selected from fluid, liquid, chilled fluid, heated fluid, chilled liquid, heated liquid, or a combination thereof.

13. The system according to claim 11 wherein the set of valves is a set of synchronized solenoid valves.

14. The system according to claim 11 wherein the controller is a centrally located computing device, computer, processor, processing device, or a combination thereof.

15. The system according to claim 11 wherein the presentation is a movie, a film, a museum exhibit, a simulation, or an on-stage production.

16. The system according to claim 11 wherein each nozzle has at least one orifice with a diameter small enough to prevent excessive wetting of an individual in an adjacent seat.

17. The system according to claim 16 wherein the system comprises two or more nozzles in one horizontal row.

18. The system according to claim 17 wherein the system comprises two or more additional nozzles in a second horizontal row adjacent to the first horizontal row.

19. The system according to claim 11 wherein each nozzle in the set of nozzles can pivot to deflect liquid in a predetermined direction.

20. A method of performing an entertainment dimension during production comprising:
spraying water sequentially from two or more nozzles in a row to simulate movement across a movie screen,
wherein the nozzles are controlled by a system comprising a pressurized source of water; a set of valves connected to the water source; the set of nozzles connected to the set of valves for expelling the water; and a controller for synchronizing with an effect the expulsion of water from the set of nozzles.
